(19) United States
(12) Patent Application Publication
(10) Pub. No.: US 2008/0193385 A1
(43) Pub. Date: Aug. 14, 2008

(54) COMPOSITIONS AND METHODS FOR TREATING NEUROPATHY

(76) Inventor: Todd Maibach, Menlo Park, CA (US)

Correspondence Address: DON J. PELTO;Sheppard, Mullin, Richter & Hampton LLP, 1300 I STREET, NW, 11TH FLOOR EAST, WASHINGTON, DC, 20005, US

(21) Appl. No.: 11/931,076

(22) Filed: Oct. 31, 2007

Related U.S. Application Data
(60) Provisional application No. 60/900,099, filed on Feb. 8, 2007.

(51) Int. Cl. A61K 31/343; A61K 9/14; A61K 9/12; A61P 25/00

(52) U.S. Cl. 424/43; 514/470; 424/489

(57) ABSTRACT

The present invention relates to compositions and methods for alleviating the painful symptoms due to neuropathy. Specifically, the method involves administering to a patient a composition comprising a nitric oxide donor that may be applied topically on the legs or arms to alleviate the negative effects due to neuropathy.

### CROSS REFERENCE TO RELATED APPLICATION

This application claims the benefit under 35 U.S.C §119, of provisional U.S. Application Ser. No. 60/900,099, filed Feb. 8, 2007, the entire and substance of which is hereby incorporated by reference.

### FIELD OF THE INVENTION

The present invention relates to compositions comprising a nitric oxide donor that may be applied topically to the skin of a patient, and methods for administering these compositions to alleviate the negative effects of patients suffering from pain due to neuropathy.

### BACKGROUND OF THE INVENTION

Neuropathy is a peripheral nerve disorder that may be genetically acquired, a result from a systemic disease, or induced by a toxic agent or other external factors. For example, a common symptom from diabetes is peripheral neuropathy. More than 15% of the 13 million diabetic patients in the United States suffer symptomatic disturbances to the nervous system such as neuropathy. Significant clinical neuropathy can develop within the first 10 years after diagnosis of diabetes and the risk of developing neuropathy increases the longer a person has diabetes. Although in most cases (30-40%) there are no symptoms, up to 60% of patients with diabetes have some form of neuropathy. Diabetic neuropathy appears to be more common in smokers, people over 40, and those who have had problems controlling their blood glucose levels.

Although methods have improved for the treatment of diabetes and its consequences, diabetic neuropathy is still an extremely serious problem. Diabetic neuropathy can be defined as a demonstrable disorder, either clinically evident or subclinical, that occurs in the setting of diabetes mellitus without other causes for peripheral neuropathy. The neuropathic disorder includes manifestations in the somatic and/or autonomic parts of the perpheral nervous system. Diabetic neuropathy often is associated with damage to the nerves just under the skin leading to one or more of the following conditions: numbness and tingling of fingers, hands, toes, and feet; weakness in hands and feet; or pain and,/or burning sensation in hands and feet.

Due to the prevalence of neuropathic pain in the arms and legs from diabetes or other diseases, there has been an interest in finding treatments where the patient can apply treatment topically. One particular way or alleviating pain due to neuropathy that has gained interest is the use of a vasodilator such as nitric oxide (NO). Research relating to the role of NO and its use as a vasodilator has been soundly established. In addition, NO is useful because there are many options for it to be manipulated in chemical compounds known as “NO donors”, where each may provide various advantages or methods of increasing the level of NO near the desired biological site. For example, some NO donors are advantageous because they can be incorporated in various delivery systems for being applied topically to the skin. One illustration is the use of isosorbide dinitrate as an NO donor in a spray delivery system (Rayman, et al., 2002, Diabetes Care 25: 1699-1703).

Due to various patient preferences and the ability to manipulate the rate of delivery of NO other delivery systems that deliver an NO donor would be useful. For example, a delivery system in a cream may be used that slows the delivery of an NO donor and hence the patient may not have to apply the active drug as frequently. Therefore, it would be beneficial to provide NO donors in numerous delivery systems in order to appeal to patients and their various preferences or needs and to maximize patient compliance.

### SUMMARY OF THE INVENTION

The present invention provides compositions and methods of using the compositions for the therapeutic treatment of pain due to neuropathy. Specifically, the present invention includes compositions comprising a nitric oxide donor to alleviate the occurrence or painful effects due to neuropathy, where the nitric oxide donor is contained within a delivery system comprising one or more from the group consisting of the form of a spray, a gel, a cream, an ointment, a balm, a foam, a paste, a solution, a transdermal patch, transdermal film, the form of a liquid in a dropper, a Snap!® delivery system, a dabomalic applicator, a bioadhesive microparticle, and in the fur of a roll-on delivery system.

In another embodiment of the present invention, the composition may comprise a nitric oxide donor or from one or more of the group consisting of L-arginine, L-citrulline, nitroglycerin (GTN), isosorbide 5-mononitrate (ISMN), isosorbide dinitrate (ISDN), pentaerythritol tetranitrate (PETN), erythrityl tetranitrate (ETN), amino acid derivatives such as N-hydroxy-L-arginine (NOHA), N.sup.6-(1-iminoethyl)lysine) (L-NIL), L-N.sup.5-(1-iminoethyl)ormithine (LN-NIO)) N.sup.a-methyl-L-arginine (L-NMMA), S-nitrosoglutathione (SNOG), S,S-dimitrosodithiol (SSDD), (N-[2-(nitroxyethyl)]-3-pyridinecarboxamide(nicorandil), sodium nitroprusside (SNP), S-nitroso-N-acetylpenicillamine (SNAP), 3-morpholino-sydnonimine (SIN-1), molsidomine, DEA-NONOate (2-(N,N-diethylamino)-diazenolate-2-oxide), spermine NONOate (N-[4-[1-(3-aminopropyl)-2-hydroxy-2-nitrosohydrazino]butyl-1,3-propanedia mine), 3-(5′-hydroxymethyl-2′furyl)-1-benzyl indazole (YC-1), 8-bromo-cyclic-GMP (8-Br-cGMP), 8-(4-chlorophenylthio)guanosine 3′,5-cyclic monophosphate (8-PCPT-cGMCP), sildenafil, cilostamide (N-cyclohexyl-N-methyl-4-(1,2-dihydro-2-oxo-6-quinolyloxy)butyramide, dipyridamole (2,6-bis(diethanol-amino)-4,8-dipipendinopyrmido-[5,4-d]pyrimidine), erythro-9-(2-hydroxy-3-nonyl)adenine (EHNA), etazolate(1-ethyl-4-[(1-methylethylidene)hydrazino]-1H-pyrazolo-[3,4-b]-pyridine-5-carboxylic acid, ethyl ester), 4-[[3,4-(methylene-dioxy)benzyl]amino]-6-chloroquinazoline (MBCQ), 8-methoxymethyl-1-methyl-3-(2-methylpropyl)xanthine (MMPX), 1-(3-chlorophenylamino)-4-phenyl-phthalazine (MY-5445), 4-(3-butoxy-4-methoxyphenyl)methyl-2-imidazolidone (Ro 20-1724), Rolipram (4-(3-(cyclopentyloxy)-4-methoxyphenyl)pyrrolidin-2-one), vinpocetine (3a,16a)-eburnamenine-14-carboxylic acid ethyl ester), zaprinast (2-propyloxyphenyl)-8-azapurin-6-one), and zardaverine (6-[4-(difluoro-methoxy)-3-methox henyl]-3(2H)-pyridazinone.

In another embodiment of the present invention, the composition may comprise one or more active drugs.

In another embodiment, the composition may comprise an angiogenesis agent. In another embodiment, the composition may comprise an angiogenesis agent from one or more of the group consisting of acidic and basic fibroblast growth factor (FGF), nibroblast growth factor (FGF-2 or FCF-1) and its denvatives, any compound that binds FGF receptors that results in receptor dimerization, autophosphorylation or subsequent activation, vascular endothelial growth factor (VEGF), any compound that results in activation of VEGF receptor-2, matrix metalloproteinase (MMP), platelet derived angiogenesis factor (PDAF), alpha5beta1 integrin, nicotine, angiogenin, Dl14 (Delta-like 4; transforming growth factor alpha (TGF-.alpha.) and beta (TGF-.beta.), tumor necrosis factor (TNF), prostaglandin, vascular permeability factor (VPF), and phospholipase C gamma 1 (PLCgamma1), Akt (PKB), PDK1, and inositol phosphate derivatives.

In another embodiment of the present invention, the composition may exclude the nitric oxide (NO) donor.

In another embodiment, the composition may exclude one or more active drugs.

In another embodiment, the composition may exclude an active drug from one or more of the group consisting of acidic and basic fibroblast growth factor (FGF), fibroblast growth factor (FGF-2 or FTF-1) and its derivatives, any compound that binds FGF receptors that results in receptor dimerization, autophosphorylation or subsequent activation, vascular endothelial growth factor (VEGF), any compound that results in activation of VEGF receptor-2, matrix metalloproteinase (MMP), platelet derived angiogenesis factor (PDAF), alpha5betal integrin, nicotine, angiogenin, Dl14 (Delta-like 4; transforming growth factor alpha (TGF-.alpha.) and beta (TOF-.beta.), tumor necrosis factor (TNT), prostaglandin, vascular permeability factor (VPF), and phospholipase C gamma 1 (PLCgamma1), Akt (PKB), PDK1, and inositol phosphate derivatives.

In another embodiment of the present invention, the composition may be administered to a patient for the treatment of nitric oxide deficiency.

In another embodiment of the present invention, the composition may be administered to a patient as a vasodilator.

In another embodiment, the composition may be administered to a patient to treat, manage the pain, and/or alleviate the occurrence or negative effects of neuropathy.

In another embodiment, the composition may be administered to a patient to treat, manage the pain, and,or alleviate the occurrence or negative effects from one of more of the group consisting of mononeuropathy, polineuropathy and mnoneuritis multiplex.

In another embodiment, the composition may be administered to a patient to treat, manage the pain, and/or alleviate the occurrence or negative effects of neuropathy when neuropathy is caused from one or more of the group consisting of diabetis, HIV-Aids, cancer, chemotherapy, celiac disease, alcoholism, infection involving a toxin, nutritional deficiencies, physical injury, polyarteritis nodosa, systemic lupus erythematosus, Sjögren's syndrome, rheumatoid arthritis, sarcoidosis, Raynaud's disease, amyloidosis, Refsum's disease, Abetalipoproteinemia, Tangier disease, Krabbe's disease, Metachromatic leukodystrophy, Fabry's disease, Dejerine-Sottas syndrome, and Charcot-Marie-Tooth Disease.

In another embodiment, the composition may be administered to a patient topically to the skin.

In another embodiment, the composition may be administered to a patient where the spray dosage form comprises one or more from the group consisting of a pump spray, an aerosol spray, and a metered dose topical aerosol.

In another embodiment, the composition may be administered to a patient where the composition is substantially free of other added active ingredients.

In another embodiment of the present invention, the compositions may comprise one or more of about 30 mg of isosorbide dinitrate; about 45 mg to about 15 mg of isosorbide dinitrate; about 37.5 mg to about 22.5 mg of isosorbide dinitrate; and about 33 mg to about 27 mg of isosorbide dinitrate.

In another embodiment of the present invention, the composition may be administered to a patient for treatment and/or pain management of neuropathy at a concentration that is about ½ to 1/20 of a concentration of a nitric oxide donor required to induce vasodilation in healthy vasculature.

In another embodiment of the present invention, the composition may be administered to a patient for treatment and/or pain management of neuropathy at a concentration that is about ¼ to 1/20 of a concentration of a nitric oxide donor required to induce vasodilation in healthy vasculature.

In another embodiment of the present invention, the composition may be administered to a patient for treatment and/or pain management of neuropathy at a concentration that is about ⅛ to 1/16 of a concentration of a nitric oxide donor required to induce vasodilation in healthy vasculature.

In another embodiment of the present invention, the methods may utilize a composition comprising a nitric oxide donor from one or more of the group consisting of L-arginine, L-citrulline, nitroglycerin (GTN), isosorbide 5-mononitrate (ISMN), isosorbide dinitrate (ISDN), pentaerythritol tetranitrate (PETN), erythrityl tetranitrate (ETN), amino acid derivatives such as N-hydroxy-L-arginine (NOHA), N.sup.6-(1-iminoethyl)lysine) (L-NIL), L-N.sup.5-(1-iminoethyl)ornithine (LN-NIO), N.sup.a-methyl-L-arginine (L-NMMA), S-nitrosoglutathione (SNOG), S,S-dinitrosodithiol (SSDD), [N-[2-(nitroxyethyl)]-3-pyridinecarboxamide (nicorandil), sodium nitroprusside (SNP), S-nitroso-N-acetylpenicillamine (SNAP), 3-morpholino-sydnonimine (SIN-1), molsidomine, DEA-NONOate (2-(N,N-diethylamino)-diazenolate-2-oxide), spermine NONOate (N-[4-[1-(3-aminopropyl)-2-hydroxy-2-nitrosohydrazino]butyl-1,3-propanedia mine), 3-(5′-hydroxymethyl-2′furyl)-1-benzyl indazole (YC-1), 8-bromo-cyclic-GMP (8-Br-cGMP), 8-(4-chlorophenylthio)guanosine 3′,5′-cyclic monophosphate (8-PCPT-cGMP), sildenafil, cilostamide (N-cyclohexyl-N-methyl-4-(1,2-dihydro-2-oxo-6-quinolyloxy)butyramide, dipyridamole (2,6-bis(diethanol-amino)-4,8-dipipendinopyrimido-[5,4-d]pyrimidine), erythro-9-(2-hydroxy-3-nonyl)adenine (EHNA), etazolate (1-ethyl-4-[(1-methylethylidene)hydrazino]-1H-pyrazolo-[3,4-b]-pyrdine-5-carboxylie acid, ethyl ester), 4-[[3,4-(methylene-dioxy)benzyl]amino]-6-chloroquinazoline (MBCQ), 8-methoxymethyl-1-methyl-3-(2-methylpropyl)xanthine (MMPX), 1-(3-chlorophenylamino)-4-phenyl-phthalazine (MY-5445), 4-(3-butoxy-4-methoxyphenyl)methyl-2-imidazolidone (Ro 20-1724), Rolipram (4-(3-(cyclopentyloxy)-4-methoxyphenyl)pyrrolidin-2-one), vinpocetine (3a,16a)-eburnamenine-14-carboxylic acid ethyl ester), zaprinast (2-propyloxyphenyl)-8-azapurin-6-one), and zardaverine (6-[4-(difluoro-methoxy)-3-methoxyphenyl]-3(2H)-pyridazinone.

In another embodiment of the present invention, the methods may utilize a composition comprising one or more active drugs.

In another embodiment, the methods may utilize a composition comprising an angiogenesis agent.

In another embodiment, the methods may utilize a composition comprising an angiogenesis agent from one or more of the group consisting of acidic and basic fibroblast growth factor (FGF), fibroblast growth factor (FGF-2 or FGF-1) and its derivatives, any compound that binds FGF receptors that results in receptor dimerization, autophosphorylation or subsequent activation, vascular endothelial growth factor (VEGF), any compound that results in activation of VEGF receptor-2, matrix metalloproteinase (MMP), platelet derived angiogenesis factor (PDAF), alpha5betal integin, nicotine, angiogenin, Dl14 (Delta-like 4; transforming growth factor alpha (TGF-.alpha.) and beta (TGF-.beta.), tumor necrosis factor (TNF), prostaglandin, vascular permeability factor (VPF), and phospholipase C gamma 1 (PLCgamma1), Akt (PKB), PDK1, and inositol phosphate derivatives.

In another embodiment of the present invention, the methods may utilize a composition that excludes a nitric oxide (NO) donor.

In another embodiment, the methods may utilize a composition that excludes one or more active drugs.

In another embodiment, the methods may utilize a composition that excludes an active drug from one or more of the group consisting of acidic and basic fibroblast growth factor (FGF), fibroblast growth factor (FGF-2 or FGF-1) and its derivatives, any compound that binds FGF receptors that results in receptor dimerization, autophosphorylation or subsequent activation, vascular endothelial growth factor (VEGF), any compound that results in activation of VEGF receptor-2, matrix metalloproteinase (MMP), platelet derived angiogenesis factor (PDAF), alpha5betal integrin, nicotine, angiogenin, Dl14 (Delta-like 4; transforming growth factor alpha (TGF-.alpha.) and beta (TGF-.beta.), tumor necrosis factor (TNF), prostaglandin, vascular permeability factor (VPF), and phospholipase C gamma 1 (PLCgamma1), Akt (PKB), PDK1, and inositol phosphate derivatives.

In another embodiment of the present invention, the methods may include methods of administering the compositions of the present invention to a patient for the treatment of nitric oxide deficiency.

In another embodiment, the methods may include methods of administering the composition of the present invention to a patient as a vasodilator.

In another embodiment, the methods may include methods of administering the present invention to a patient to treat, manage the pain, and or alleviate the occurrence or negative effects of neuropathy.

In another embodiment, the methods may include methods of administering the present invention to a patient to treat, manage the pain and/or alleviate the occurrence or negative effects from one of more of the group consisting of mononeuropathy, polineuropathy and mononeuritis multiplex.

In another embodiment, the methods may include methods of administering the present invention to a patient to treat, manage the pain, and/or alleviate the occurrence or negative effects of neuropathy when neuropathy is caused from one or more of the group consisting of group consisting of diabetis, HIV-Aids, cancer, chemotherapy, celiac disease, alcoholism, infection involving a toxin, nutritional deficiencies, physical injury, polyarteritis nodosa, systemic lupus erythematosus, Sjögren's syndrome, rheumatoid arthritis, sarcoidosis, Raynaud's disease, amyloidosis. Refsum's disease, Abetalipoproteinemia, Tangier disease, Krabbe's disease, Metachromatic leukodystrophy, Fabry's disease, Dejerine-Sottas syndrome, and Charcot-Marie-Tooth Disease. In another embodiment, the methods may include methods of administering the present invention to a patient topically on the skin.

In another embodiment, the methods may include methods of administering the present invention to a patient where the spray dosage form comprises one or more from the group consisting of a pump spray, an aerosol spray, and a metered dose topical aerosol.

In another embodiment of the present invention, the methods may utilize compositions substantially free of other added active ingredients.

In another embodiment of the present invention, the methods may utilize compositions comprising one or more of about 30 mg of isosorbide dinitrate; about 45 mg to about 15 mg of isosorbide dinitrate; about 37.5 mg to about 22.5 mg of isosorbide dinitrate; and about 33 mg to about 27 mg of isosorbide dinitrate.

In another embodiment of the present invention, the composition may comprise a delivery system configured such that a nitric oxide (NO) donor only contacts the skin at the site of administration.

In another embodiment of the present invention, the methods may utilize composition comprising a delivery system configured such that a nitric oxide (NO) donor only contacts the skin at the site of administration.

In another embodiment, the methods may include methods of administering the present invention to a patient for treatment and/or pain management of neuropathy at a concentration that is about ½ to 1/20 of a concentration of a nitric oxide donor required to induce vasodilation in healthy vasculature.

In another embodiment, the methods may include methods of administering the present invention to a patient for treatment and/or pain management of neuropathy at a concentration that is about ¼ to 1/20 of a concentration of a nitric oxide donor required to induce vasodilation in healthy vasculature.

In another embodiment, the methods may include methods of administering the present invention to a patient for treatment and/or pain management of neuropathy at a concentration that is about ⅛ to 1/16 of a concentration of a nitric oxide donor required to induce vasodilation in healthy vasculature.

### DETAILED DESCRIPTION OF THE INVENTION

Neuropathy is a peripheral nerve disorder that may be induced from various sources. For instance, peripheral neuropathies can be genetically acquired, can result from a systemic disease, or can be induced by a toxic agent or other external factors.

External factors that induce neuropathy may include alcoholism, certain B-vitamin deficiencies, and chemotherapeutic agents such as platinum based agents. For example, chemotherapeutic agents known to cause sensory and/or motor neuropathies include vincristine, an antineoplastic drug used to treat haematological malignancies and sarcomas. The neurotoxicity is dose-related, and exhibits as reduced intestinal motility and peripheral neuropathy, especially in the distal muscles of the hands and feet, postural hypotension, and atony of the urinary bladder. Similar problems have been documented with taxol and cisplatin (Mollman, J. E, 1990, New Eng Jour Med. 322:126-127), although cisplatin-related neurotoxicity can be alleviated with nerve growth factor (NGF) (Apfel, S. C. et al., 1992, Annals of Neurology 31:76-80). Although the neurotoxicity is sometimes reversible after removal of the neurotoxic agent, recovery can be a very slow process (Legha, S., 1986, Medical Toxicology 1:421-427 Olesen, et al., 1991, Drug Safety 6:302-314).

There are also a number of inherited peripheral neuropathies, including: Refsum's disease. Abetalipoproteinemia Tangier disease, Krabbe's disease, Metachromatic leukodystrophy, Fabry's disease Dejerine-Sottas syndrome, and others. Of all the inherited neuropathies, the most common by far is Charcot-Marie-Tooth Disease.

Charcot-Marie-Tooth (CMT) Disease (also known as Peroneal Muscular Atrophy, or Hereditary Motor Sensory Neuropathy (HMSN)) is the most common hereditary neurological disorder. It is characterized by weakness and atrophy, primarily of the peroneal muscles, due to segmental demyelination of peripheral nerves and associated degeneration of axons and anterior horn cells. Autosomal dominant inheritance is usual, and associated degenerative CNS disorders, such as Friedreich's ataxia, are common.

Diabetic neuropathy is one of the most common examples of disease-induced peripheral neuropathy. Similar neuropathies can occur in conditions such as acromegaly, hypothyroidism, AIDS, leprosy, Lyme disease, systemic lupus erythematosus, rheumatoid arthritis, Sjogren's syndrome, periarteritis nodosa, Wegener's granulomatosis, cranial arteritis, and sarcoidosis, among other conditions.

Although methods have improved for the treatment of diabetes and its consequences, diabetic neuropathy is still an extremely serious problem. Diabetic neuropathy can be defined as a demonstrable disorder, either clinically evident or subclinical, that occurs in the setting of diabetes mellitus without other causes for peripheral neuropathy. The neuropathic disorder includes manifestations in the somatic and/or autonomic parts of the peripheral nervous system. Diabetic neuropathy often is associated with damage to the nerves just under the skin leading to one or more of the following conditions: numbness and tingling of fingers, hands, toes, and feet, weakness in hands and feet; or pain and/or burning sensation in hands and feet. Nerve damage as the result of peripheral neuropathy can also lead to problems with the GI tract, heart, and sexual organs, causing indigestion diarrhea or constipation, dizziness, bladder infections, and impotence.

More than 15% of the 13 million diabetic patients in the United States suffer symptomnatic disturbances to the nervous system Significant clinical neuropathy can develop within the first 10 years after diagnosis of diabetes and the risk of developing neuropathy increases the longer a person has diabetes. Although in most cases (30-40%) there are no symptoms, up to 60% of patients with diabetes have some form of neuropathy. Diabetic neuropathy appears to be more common in smokers, people over 40, and those who have had problems controlling their blood glucose levels.

Due to the many forms and causes of neuropathy, there are various therapeutic strategies to treat or alleviate the pain induced by neuropathy. One particular method for alleviating pain due to neuropathy that has drawn particular interest is the chemical compound, nitric oxide (NO). The biological importance of NO is well documented. In mammals, NO is an endogenous physiological mediator of many biological functions. In addition, it is applied pharmacologically in various forms usually referred to as NO donors (nitroglycerin, sodium nitroprusside, etc.) to correct NO deficient states or to regulate the activities of many tissues. Topical applications may be used to help wound and burn healing, hair growth, impotence, and cause vasodilatation where needed (e.g., ripening of the cervix in pregnancy). U.S. Pat. No. 5,519,020 relates to polymeric nitric oxide sources thought to be useful to promote healing.

Moreover, NO generation may play an important role in diabetic neuropathic pain. Hence, there are has been particular interest in NO donors that may be effective in relieving pain associated with diabetic neuropathy or other causes of neuropathy. However, NO donors can come in many forms and compounds. The present invention includes formulations and compounds that are defined herein as a nitric oxide donor.

Herein, nitric oxide donor is intended to mean any compound which mimics the effects of NO, generates or releases NO through biotransformation, any compound which generates NO spontaneously, any compound which spontaneously releases NO, or any compound which in any other manner generates NO or a NO-like moiety when administered to a mammal. Such a compound can also be referred to as a “NO mimic,” “NO prodrug,” “NO producing agent,” “NO delivering compound,” “NO generating agent,” and “NO provider,” Examples of such compounds include, but are not necessarily limited to metabolic precursors for NO such as L-arginine and L-citrulline: so-called “organonitrates” such as nitroglycerin (GTN), isosorbide 5-mononitrate (ISMN), isosorbide dinitrate (ISDN) pentaerythritol tetranitrate (PETN), erythrityl tetranitrate (ETN) amino acid derivatives such as N-hydroxy-L-arginine (NOHA), N.sup.6-(1-iminoethyl)lysine) (L-NIL), L-N.sup.5-(1-minoethyl)omithine (LN-NIO), N.sup.a-methyl-L-arginine (L-NMMA), and S-nitrosoglutathione (SNOG); other compounds which generate or release NO under physiologic conditions such as S,S-dinitrosodithiol (SSDD), [N-[2-(nitroxyethyl)]-3-pyridinecarboxamide(nicorandil), sodium nitroprusside (SNP), S-nitroso-N-acetylpenicillamine (SNAP), 3-morpholino-sydnonimine (SIN-1), molsidomine, DEA-NONOate (2-(N,N-diethylamino)-diazenolate-2-oxide), spermine NONOate (N-[4-[1-(3-aminopropyl)-2-hydroxy-2-nitrosohydrazino]butyl-1,3-propanedia mine), and NO gas, or a functional equivalent thereof. The organic nitrates GTN ISMN, ISDN, ETN and PETN, as well as nicorandil are commercially available in pharmaceutical dosage forms. SIN-1, SNAP, S-thioglutathione, L-NMMA, L-NIL, L-NIO, spermine NONOate, and DEA-NONOate are commercially available from Biotium, Inc. 183 Shoreline Court, Richmond, Calif., USA. The term “NO donor” or more specifically a “NO-mimetic,” as used herein, is also intended to mean any compound that acts as a NO pathway mimetic, that has NO-like activity, or that mimics the effect of NO, e.g., CO. Such compounds do not necessarily release, generate, or provide NO, but they have the same effect as NO on a pathway that is affected by NO. For example, NO can have both cyclic GMP-dependent and cyclic GMP-independent effects. NO is known to activate the soluble form of guanylyl cyclase thereby increasing intracellular levels of the second messenger cyclic GMP. As such, any compounds which directly activate guanylyl cyclase such as 3-(5′-hydroxethyl-2′furyl)-1-benzyl indazole (YC-1) or which act as cyclic-GMP analogues such as 8-bromo-cyclic-GMP (8-Br-cGMP) and 8-(4-chlorophenylthio)guanosine 3′,5′-cyclic monophosphate (8-PCPT-cGMP) are considered NO-mimetics. For purposes of the present invention, phosphodiesterase inhibitors or any compound that inhibits enzymatic degradation of a cyclic nucleotide are also considered NO-mimetics. These NO-mimeties include, for example, compounds that antagonize or inhibit the biosynthesis or actions of any enzyme that degrades a cyclic nucleotide. Such degradation may comprise the cleavage of a phosphodiester such as cGMP or cAMP to give a phosphomonoester and a free hydroxyl group. Examples of these NO-mimetics include, but are not limited to, sildenafil; cilostamide (N-cyclohexyl-N-methyl-4-(1,2-dihydro-2-oxo-6-quinolyloxy)butytamide; dipyridamole(2,6-bis(diethanol-amino)-4,8-dipipendinopyrimido-[5,4-d]primidine); erythro-9-(2-hydroxy-3-nonyl)adenine (EHNA); etazolate(1-ethyl-4-[(1-methylethylidene)hydrazino]-1H-pyazolo-[3,4-b]-pyridine-5-carboxylic acid, ethyl ester); 4-[[3,4-(methylene-dioxy)benzyl]amino]-6-chloroquinazoline (MBCQ); 8-methoxymethyl-1-methyl-3-(2-methylpropyl)xanthine (MMPX; 1-(3-chlorophenylamino)-4-phenyl-phthalazine (MY-5445); 4-(3-butoxy-4-methoxyphenyl)methyl-2-imidazolidone (Ro 20-1724); Rolipram (4-(3-(cyclopentyloxy)-4-methoxyphenyl)pyrrolidin-2-one); vinpocetine (3a-16a)-ebumamenine-14-carboxylic acid ethyl ester); zaprinast(2-propyloxyphenyl)-8-azapurin-6-one); and zardaverine(6-[4-(difluoro-methoxy)-3-methoxyphenyl]-3(2H)-pydazinone. Such NO-mimetics are available, for example, from Tocris Cookson Inc. (Ballwin, Mo.).

Another particular strategy to assist in the treatment or alleviation of pain induced by neuropathy would be the use of agents that activate or promote angiogenesis. Angiogenesis is the formation of new blood vessels and is essential for embryonic development, subsequent growth, tissue repair and wound healing. Specifically, angiogenesis is the process by which new blood vessels are formed from extant capillaries by encouraging the proliferation of endothelial cells. Endothelial cells line the walls of blood vessels; capillaries are comprised almost entirely of endothelial cells. Therapeutic angiogenesis by introduction of angiogenic agents would therefore be a method to relieve inadequate blood flow by the directed growth and proliferation of blood vessels. Lederman et al., 2003, British Journal of Pharmacology 140: 637-46. Hence, increasing blood flow and growth of blood vessels would be beneficial in the development of small nerves and a counterbalance to the adverse effects due to neuropathy.

Angiogenesis agents have a wide range of different properties and mechanisms of action and can come in many forms and compounds. The present invention includes formulations and compounds that are defined herein as an angiogenesis agent.

Herein, aniogenesis agent is intended to mean any compound that promotes or assists either directly or indirectly in the growth of blood vessels or involved in vasculogenesis. Examples of such compounds include, but are not necessarily limited to acidic and basic fibroblast growth factor (FGF), basic fibroblast growth factor (FGF-2 or FGF-1) and its derivatives; any compound that binds FGF receptors that results in receptor dimerization, autophosphorylation or subsequent activation: vascular endothelial growth factor (VEGF); any compound that results in activation of VEGF receptor-2; matrix metalloproteinase (MMP); platelet derived angriogenesis factor (PDAF); alpha5betal integrin; nicotine; angiogenin; Dl14 (Delta-like 4; transforming growth factor alpha (TGF-.alpha.) and beta (TGF-.beta.); tumor necrosis factor (TNF); prostaglandin; vascular permeability factor (VPF); and phospholipase C gamma 1 (PLCgamma1).

Angiogenesis agents may be agents that have a short term or long term effect for the promotion of blood vessel formation, Angiogenesis agents may be those that activate signaling pathways that promote angiogenesis or inhibit signaling pathways with any apoptotic potential. For example, an angiogenesis agent may be a drug that induces NO syntheses by activating the PI3/Akt pathway which promotes endothelial cell growth or survival. Therefore, angiogenesis agents may include Akt (PKB), PDK1 or inositol phosphate derivatives that activate or assist in activation of the PI3/Akt pathway.

Due to various patient preferences in the application of a particular drug and the various advantages that delivery systems provide, it is beneficial to have a particular drug available in many delivery systems. For example, an NO donor in a spray delivery system was recently established (Rayman, et al. (2002) Diabetes Care 25: 1699-1703). However, a patient may prefer a delivery system wherein the active drug can be applied to the skin by a lotion or ointment. Moreover, applying a drug in an ointment or lotion delivery may be able to take advantage of a slower delivery of the active drug. Hence, the patient may not have to apply the active drug as frequently to relieve painful symptoms due to neuropathy.

Delivery systems containing the composition of the invention may be formulated in any pharmaceutical state suitable for topical application, examples of which include liquid, aerosol, thickened liquid emulsion, semisolid and powder. Specifically, the delivery system may be a spray, a roll on, a gel, a cream, an ointment a balm a transdermal patch, transdermal film, a Snap!® delivery system, the form of a liquid in a dropper, a dabomalic applicators, a bioadhesive microparticle, a foam, a paste, a solution and so forth.

The methods of the invention may employ any of such formulations as may be appropriate for treatment in particular cases. Advantageously, the composition can be formulated into highly convenient dosage forms with thickening agents to include thickened solutions or lotions, ointments to include creams and gels, and so forth.

Thickened solutions or lotions and ointments may be formed by incorporating with the active ingredients various gelling agents or other thickeners (viscosity increasers) which permit release of the active ingredients to the skin or tissue upon or following application. These forms are advantageously employed to lessen the runoff from the skin or tissue which may occur with more fluid (less viscous) formulations. Importantly, they also permit more sustained contact of the active ingredient(s) and any penetration enhancer with the treated surfaces, thus permitting an enhancement of the speed of delivery of the active ingredient(s) subcutaneously, and providing more accurate and controllable dosing. Accidental spilling and undesired contact with the composition can also be minimized with such types of formulations.

It can be advantageous to employ water-dispersible thickening agents, i.e., agents dispersible in water to form a homogeneous distribution or even solution, such as the polyethylene glycols and similar agents, as they are readily compatible with water or other diluents which may be formulated in the composition. Alternatively, an emulsion base may be employed to impart the desired thickening effect, together with the emollient effect of the lipoid phase of the emulsion base.

Water-soluble or water-dispersible thickening bases or substances may employ polyethylene glycols and the like of different viscosities depending upon the desired consistency and concentration of active ingredient(s) which may be incorporated into the composition. Other thickening agents which may be suitable for employment herein include but are not limited to water-dispersible gums, carboxyvinyl polymers, methyl cellulose, sodium carboxymethyl cellulose, and alginates.

Lotions and ointments incorporating emulsion bases may contain the usual ingredients to provide the base including fatty alcohols such as acetyl alcohol, an emulsifier such as, for example, lauryl sulfate, and water. Also, the remainder of a topical preparation may contain one or more conventional ointment components such as, for example, white petroleum, lanolin, distilled water and mineral oil in conventional amounts.

In another embodiment of the invention, a drug delivery system may be used that allows the active drug, specifically a nitric oxide (NO) donor, to be applied topically to the desired biological site of the patient without contacting the hands during application. For example, a roll-on drug delivery system that is similar to a typical deodorant applicator may be used. Active drugs that are applied to a non-symptomatic location of the patient may exhibit unwanted pharmacological responses. For instance, the active drug nitric oxide often causes adverse side affects such as headaches due to non-specific delivery or over application. Therefore, a delivery system that allows the patient or caregiver to not touch the active drug with his or her hands during application may lower adverse side effects to the hands or other non-designated areas of the body.

In another embodiment of the invention, a nitric oxide donor may be administered to the patient by a drug delivery system in minimal doses or microdoses, so as to provide dosages which are about one half to about one twentieth (½ to 1/20) of those known to induce vasodilation in healthy vasculature or non-symptomatic locations of the patient. The low doses of a nitric oxide donor effectively enhance NO and alleviate vascular conditions due to neuropathy without inducing undesirable side effects such as systemic vasodilation or headaches. The range of about ½ to about 1/20 is derived from the observation that at doses which are below about ½ the normal dose, undesirable side effects are generally no longer seen. At about 1/20 the normal dose, however, the desired effect is also generally no longer observed. See, for example, U.S. Pat. Nos. 6,165,975 and 6,610,652, which are expressly incorporated by reference herein.

In another embodiment of the invention, a single dose delivery system such as SNAP!® (Minneapolis, Minn.) may be used. SNAP!® is a convenient method for delivering the active drug in the form such as a cream, gel, paste, lotion or in the form of a strip or sponge that contains a foam. Such convenience is provided by its single use packaging and its easy-to-use dispensing method that requires only one hand. By being contained in a single use system, the active drug volume can be more consistent when applied for each treatment. Moreover neuropathic patients who have difficulties unscrewing bottles or tops for ointments or gels due to severe symptoms in their hands will have the option of an easy-to-use system for opening and applying the active drug.

In another embodiment of the invention, liposomes, also known as vesicles, may be used. Liposomes function as carriers whose essential structural feature is a bipolar lipid membrane which envelops an aqueous core volume in which pharmacological agents are solubilized and therefore encapsulated. Liposomal encapsulated drugs have shown promise in treating diseases and have performed as diagnostic tools for the early detection of cancer and other maladies. As a result, liposomes have shown potential as site-specific carrier systems for a variety of therapeutic agents including enzymes for enzyme replacement therapy, hormones, cell modifying agents and genetic material. The pharmaceutical products which have been delivered to designated sites in vivo have demonstrated an improvement in therapeutic indices. Thus, by using liposomes for site-specific delivery, the results show a general lowering of adverse side effects as lower overall doses of therapeutic agents are administered. Agents that are delivered in a conventional or non-specific manner often spread or are dispersed to non-designated areas and thus exhibit adverse side effects and unwanted pharmacological responses.

In one embodiment, the delivery system can be a controlled release drug delivery system of a suitable biocompatible excipient for applying the agent to include a lipophilic carrier or a hydrophilic carrier. An example of a suitable carrier is a lipophilic carrier such as semi-synthetic glycerides of saturated fatty acids. Non-limiting examples of a hydrophilic carrier include polyethylene glycol having an average molecular weight of 6000, polyethylene glycol having an average molecular weight of 1500, polyethylene glycol having an average molecular weight of 400 or mixtures thereof. The biocompatible excipient can also include a muco-adhesive agent such as alginate, pectin, or cellulose derivative. The biocompatible excipient can also include a penetration enhancer such as bile salts, organic solvents, ethoxydiglycol, or interesterified stone oil.

In one embodiment of the invention the excipient comprises between about 60 to 90% by weight lipophilic earrer between about 5 to 25% muco-adhesive agent, and between about 5 to 20% penetration enhancer,

In another embodiment of the invention, the excipient comprises between about 60 to 90% bv weight hydrophilic carrier, between about 5 to 25% mucc-adhesive agent, and between about 5 to 20% penetration enhancer.

In another embodiment of the invention, the drug delivery system comprises a standard fragrance free lotion formulation, for example that sold under the trademark Jergens® Lotion.

In another embodiment, the biocompatible excipient can include glycerin, mineral oil, polycarbophil, carbomer 934P, hydrogenated palm oil, glyceride, sodium hydroxide, sorbic acid, and purified water.

In order to achieve desirable drug release, the active ingredient will be incorporated into an excipient (i.e., vehicle or carrier) for which the drug has low affinity. Hence, hydrophilic drugs will be incorporated into lipophilic carriers and lipophilic drugs will be incorporated into hydrophilie carriers.

Preferred lipophilic carriers for use with hydrophilic drugs, include semi-synthetic glycerides of saturated fatty acids, particularly from C8 to C18, such as SUPPOCIRE.RTM. AS2 (Gattefosse Inc., Westwood, N.J.).

Preferred hydrophilic carriers, for promoting synergistic drug delivery, include polyethylene glycol or mixtures thereof, such as PEG 6000/PEG 1500, or PEG 6000/PEG 1500/PEG 400, or PEG 6000/PEG 400 (Sigma/Aldrich, St. Louis, Mo.).

To slow the NO generation, one may prepare an ointment from a nonaqueous medium (e.g., petroleum jelly or petroleum) and powdered ingredients, which, on being applied topically on the skin, will release NO as water permeates through this medium. Alternatively, one may convert the aqueous sodium nitrite solution into an aqueous gel with hydroxyethylcellulose (or other gel-forming substance or compound) and combine this gel with another gel obtained from aqueous ascorbic and maleic acids with hydroxyethylcellulose for topical application (on intact skin, burns, intra-cavity, etc.). The two gels may be admixed immediately before use (possibly from a single container with separate chambers and dual nozzle, via pushing or squeezing the two gels through the nozzle of a device), or may be applied in as a transdermal patch for further slowing down the delivery of NO.

A example of a suitable system is a standard fragrance free lotion formulation containing glycerol, ceramides, mineral oil, petroleum, parabens, fragrance and water such as the product sold under the trademark Jergens®. (Andrew Jergens Co., Cincinnati, Ohio). This formulation was used by Hargrove et al. (Abstract No. 97,051, North American Menopause Society, Boston, Mass., September 1997) for transcutaneous delivery of estradiol and progesterone. Suitable nontoxic pharmaceutically acceptable systems for use in the compositions of the present invention bill he apparent to those skilled in the art of pharmaceutical formulations and examples are described in REMINGTON'S PHARMACEUTICAL SCIENCES, 19th Edition, A. R. Gennaro, ed., 1995.

Other objectives, features and advantages of the present invention will become apparent from the following specific examples. The specific examples, while indicating specific embodiments of the invention, are provided by way of illustration only. Accordingly, the present invention also includes those various changes and modifications within the spirit and scope of the invention that may become apparent to those skilled in the art from this detailed description. The invention will be further illustrated by the following non-limiting examples.

### EXAMPLES

Without requiring further elaboration, it is believed that one skilled in the art, using the preceding description, can utilize the present invention to the fullest extent. The following examples are illustrative only, and not limiting of the remainder of the disclosure in any way whatsoever.

### Example 1

An ointment was prepared by admixing 12.5 g of 4 percent isosorbide dinitrate in white petroleum, lanolin, and distilled water with 37.5 g white petroleum, USP (VASELINE; Chesebrough-Ponds USA Co., Greenwich, Conn.) in a laboratory mixing vessel at room temperature. The resulting mixture was 50 g of a 1.0 percent isosorbide dinitrate ointment.

### Example 2

A study is undertaken to evaluate the effectiveness of the compositions of the present invention in the treatment of patients. The objective of the study is to determine whether topical treatment of the compositions of the present invention results in a rapid improvement of the symptoms of diabetic neuropathy.

A double-blind, placebo controlled study is conducted over a 4 week period. A total of 120 subjects, all presenting for pain relief from symptoms of diabetic neuropathy located in the feet are chosen for the study. The patients range in age from 18 to 65 years old.

An initial assessment of the symptoms of each patient is conducted when the patients initially present for treatment. The patient rates the severity of the symptoms on a 4-point scale (0: absent; 1:mild; 2: moderate; 3: severe). For inclusion in the study a patient must be rated with a score of two or above for painful neuropathy in the feet.

The 120 subjects chosen for the study are separated into two separate groups of 60. The characteristics of the symptoms between the two groups are comparable. The first group administers approximately 2500 to 3000 mg of 1.0 percent isosorbide dinitrate ointment as in Example 1 by applying with the finger topically to the feet four times daily. The second group administers a placebo medication that is similar in all respects to the administered composition in Example 1 except for the exclusion of the active ingredient isosorbide dinitrate, by applying with the finger topically to the feet four times daily.

All patients were interviewed and examined 1, 2, 3 and 4 weeks after initiating the treatment using the same 4 point scale. The symptoms evaluated are overall neuropathic pain in the feet and burning sensation in the feet.

The assessment of the relief for overall pain severity and burning sensation is conducted for each subject group. The data is evaluated using multiple linear regression analysis and a standard t-test. In each analysis, the baseline value of the outcome variable is included in the model as a covariant. Treatment by covariant interaction effects is tested by the method outlined by Weigel & Narvaez, 12 CONTROLLED CLINICAL TRIALS 378-94 (1991). If there are no significant interaction effects, the interaction terms are removed from the model. The regression model assumptions of normality and homogeneity of variance of residuals are evaluated by inspection of the plots of residuals versus predicted values. Detection of the temporal onset of effects is done sequentially by testing for the presence of significant treatment effects at 1 2, 3 and 4 weeks, proceeding to the earlier time in sequence only when significant effects have been identified at each later time period. Changes form the baseline within each group are evaluated using paired t-tests. In addition, analysis of variance is performed on all baseline measurements and measurable subject characteristics to assess homogeneity between groups. All statistical procedures are conducted using the Statistical Analysis System (SAS Institute Inc., Cary, N.C.). An alpha level of 0.05 is used in all statistical tests.

While specific embodiments of the present invention have been described, other and further modifications and changes may be made without departing from the spirit of the invention. All further and other modifications and changes are included that come within the scope of the invention as set forth in the claims. The disclosures of all publications cited above are expressly incorporated by reference in their entireties to the same extent as if each were incorporated by reference individually.

What is claimed is:

1. A composition comprising a nitric oxide (NO) donor administrable to a patient, wherein said nitric oxide donor is contained within a delivery system comprising one or more of the group consisting of a spray, a gel, a cream, an ointment, a balm, a foam, a paste, a solution, a transdermal patch, a transdermal film, a liquid in a dropper, a Snap!® delivery system, a dabomalic applicator, a bioadhesive microparticle, and a roll-on delivery system.

2. The composition of claim 1, wherein said nitric oxide donor is selected from one or more of the group consisting of L-arginine, L-citrulline, nitroglycerin (GTN), isosorbide 5-mononitrate (ISMN), isosorbide dinitrate (ISDN), pentaerythritol tetranitrate (PETN), erythrityl tetranitrate (ETN), amino acid derivatives such as N-hydroxy-L-arginine (NOHA), N-.sup.6-(1-iminoethyl)lysine) (L-NIL), L-N.sup.5-(1-iminoethyl)ornithine (LN-NIO), N.sup.a-methyl-L-arginine (L-NMMA), S-nitrosoglutathione (SNOG), S,S-dinitrosodithiol (SSDD), [N-[2-(nitroxyethyl)]-3-pyridinecarboxamide (nicorandil), sodium nitroprusside (SNP), S-nitroso-N-acetylpenicillamine (SNAP), 3-morpholino-sydnonimine (SIN-1), molsidomine, DEA-NONOate (2-(N,N-diethylamino)-diazenolate-2-oxide), spermine NONOate (N-[4-[1-(3-aminopropyl)-2-hydroxy-2-nitrosohydrazino]butyl-1,3-propanedia mine), 3-(5′-hydroxyethyl-2′furyl)-1-benzyl indazole (YC-1), 8-bromo-cyclic-GMP (8-Br-cGMP), 8-(4-chlorophenylthio)guanosine 3′,5′-cyclic monophosphate (8-PCPT-cGMP), sildenafil, cilostamide (N-cyclohexyl-N-methyl-4-(1,2-dihydro-2-oxo-6-quinolyloxy)butyramide, dipyridamole(2,6-bis(diethanol-amino)-4,8-dipipendinopyrimido-[5,4-d]pyrimidine), erythro-9-(2-hydroxy-3-noyl)adenine (EHNA), etazolate(1-ethyl-4-[(1-methylethylidene)hydrazino]-1H-pyrazolo-[3,4-b]-pydine-5-carboxylie acid, ethyl ester), 4-[[3,4-(metlylene-dioxy)benzyl]amino]-6-chloroquinazoline (NBCQ), 8-methoxymethyl-1-methyl-3-(2-methylpropyl)xanthine (MMPX), 1-(3-chlorophenylamino)-4-phenyl-phthalazine (MY-5445), 4-(3-butoxy-4-methoxyphenyl)methyl-2-imidazolidone (Ro 20-1724), Rolipram (4-(3-(cyclopentyloxy)-4-methoxyphenyl)pyrrolidin-2-one), vinpoectine (3a,16a)-eburnamenine-4-carboxylic acid ethyl ester), zaprinast(2-propyloxyphenyl)-8-azapurin-6-one), and zardaverine(6-[4-(difluoro-methoxy)-3-methoxyphenyl]-3(2H)-pyridazinone.

3. The composition of claim 1 wherein said composition comprises one or more active drugs.

4. The composition of claim 3, wherein said active drug is an angiogenesis agent.

5. The composition of claim 4, wherein said angiogenesis agent is selected from one or more of the group consisting of acidic and basic fibroblast growth factor (FGF), fibroblast growth factor (FGF-2 or FGF-1) and its derivatives, any compound that binds FGF receptors that results in receptor dimerization, autophosphorylation or subsequent activation, vascular endothelial growth factor (VEGF), any compound that results in activation of VEGF receptor-2, matrix metalloproteinase (MMP), platelet derived angiogenesis factor (PDAF), alpha5beta1 integrin, nicotine, angiogenin, Dl14 (Delta-like 4; transforming growth factor alpha (TCC-.alpha.) and beta (TGF-.beta.), tumor necrosis factor (TNF), prostaglandin, vascular permeability factor (VPF), and phospholipase C gamma 1 (PLCgamma1), Akt (PKB), PDK1, and inositol phosphate derivatives.

6. The composition of claim 1, wherein said nitric oxide donor is administrable to said patient for treatment of nitric oxide deficiency.

7. A composition comprising claim 1, wherein said composition is administrable to said patient as a vasodilator.

8. The composition of claim 7, wherein said composition is administrable to said patient for treatment and/or pain management of neuropathy.

9. The composition of claim 8, wherein said neuropathy is from one or more of the group consisting of mononeuropathy, polineuropathy and mononeuritis multiplex.

10. The composition of claim 9, wherein said neuropathy is associated with one or more of the group consisting of diabetis, HIV-AIDS, cancer, chemotherapy, celiac disease, alcoholism, infection involving a toxin, nutritional deficiencies. physical injury, polyarteritis nodosa, systemic lupus erythematosus, Sjögren's syndrome, rheumatoid arthritis, sarcoidosis, Raynaud's disease, amyloidosis, Refsum's disease. Abetalipoproteinmia, Tangier disease Krabbe's disease, Metachromatic leukodystrophy, Fabry's disease, Dejerine-Sottas syndrome, and Charcot-Marie-Tooth Disease.

11. The composition of claim 1, wherein said composition is administered to said patient topically to the skin.

12. The composition of claim 1, wherein said spray is one or more of the group consisting of a pump spray, an aerosol spray and a metered dose topical aerosol.

13. The composition of claim 8, wherein said composition is administrable to said patient at about ½ to about 1/20 of a concentration of said nitric oxide donor required to induce vasodilation in healthy vasculature.

14. A method comprising administering the composition of claim 1 to a patient.

15. The method of claim 14, wherein said nitric oxide donor is selected from one or more of the group consisting of L-arginine, L-citrulline, nitroglycerin (GTN), isosorbide 5-mononitrate (ISMN), isosorbide dinitrate (ISDN), pentaerythritol tetranitrate (PETN), erythrityl tetranitrate (ETN), amino acid derivatives such as N-hydroxy-L-arginine (NOHA), N.sup.6-(1-iminoethyl)lysine) (L-NIL), L-N.sup.5-(1-iminoethyl)ornithine (LN-NIO), N.sup.a-methyl-L-arginine (L-NMMA), S-nitrosoglutathione (SNOG), S,S-dinitrosodithiol (SSDD), [N-[2-(nitroxyethyl)]-3-pyridinecarboxamide(nicorandil), sodium nitroprusside (SNP), S-nitroso-N-acetylpenicillamine (SNAP), 3-morpholino-sydnonimine (SIN-1), molsidomine, DEA-NONOate (2-(N,N-diethylamino)-diazenolate-2-oxide), spernine NONOate (N-[4-[1-(3-aminopropyl)-2-hydroxy-2-nitrosohydrazino]butyl-1,3-propanedia mine), 3-(5′-hydroxyethyl-2′furyl)-1-benzyl indazole (YC-1), 8-bromo-cyclic-GMP (8-Br-cGMP), 8-(4-chlorophenylthio)guanosine 3′,5′-cyclic monophosphate (8-PCPT-cGMP), sildenafil cilostamide (N-cyclohexyl-N-methyl-4-(1,2-dihydro-2-oxo-6-quinoylyoxy)butyramide, dipyridamole (2,6-bis(diethanol-amino)-4,8-dipipendinopyrimido-[5,4-d]pyrimidine), erythro-9-(2-hydroxy-3-nonyl)adenine (EHNA), etazolate(1-ethyl-4-[(1-methylethylidene)hydrazino]-1H-pyrazolo-[3,4-b]-pyridine-5-carboxylic acid, ethyl ester), 4-[[3,4-(methylene-dioxy)benzyl]amino]-6-chloroquinazoline (MBCQ), 8-methoxymethyl-1-methyl-3-(2-methylpropyl)xanthinc (MMPX), 1-(3-chlorophenylamino)-4-phenyl-phthalazine (MY-5445), 4-(3-butoxy-4-methoxyphenyl)methyl-2-imidazolidone (Ro 20-1724), Rolipram(4-(3-(cyclopentyloxy)-4-methoxyphenyl)pyrrolidin-2-one), vinpocetine(3a,16a-eburnamenine-14-carboxylic acid ethyl ester), zaprinast(2-propyloxyphenyl)-8-azapurin-6-one), and zardaverine(6-[4-(difluoro-methoxy)-3-methoxyphenyl]-3(2H)-pyridazinone.

16. The method of claim 14, wherein said composition comprises one or more active drugs.

17. The method of claim 16, wherein said active drug is an angiogenesis agent.

18. The method of claim 17, wherein said angiogenesis agent is selected from one or more of the group consisting of acidic and basic fibroblast growth factor (FGF), fibroblast growth factor (FGF-2 or FGF-1) and its derivatives, any compound that binds FGF receptors that results in receptor dimenrzation, autophosphorylation or subsequent activation, vascular endothelial growth factor (VEGF), any compound that results in activation of VEGF receptor-2, matrix metalloproteinase (MMP), platelet derived angiogenesis factor (PDAF), alpha5beta1 integrin, nicotine, angiogenin, Dl14 (Delta-like 4; transforming growth factor alpha (TGF-.alpha.) and beta (TGF-.beta.), tumor necrosis factor (TNF), prostaglandin, vascular permeability factor (VPF), and phospholipase C gamma 1 (PLCgamma1), Akt (PKB), PDK1, and inositol phosphate derivatives.

19. The method of claim 14, wherein said nitric oxide donor is administrable to said patient for treatment of nitric oxide deficiency.

20. The method of claim 14, wherein said nitric oxide donor is administrable to said patient for treatment and/or pain management of neuropathy.

21. The method of claim 20, wherein said neuropathy is from one or more of the group consisting of mononeuropathy, polineuropathy and mononeuritis multiplex.

22. The method of claim 21, wherein said neuropathy is caused from one or more of the group consisting of diabetis, HIV-AIDS, cancer, chemotherapy, celiac disease, alcoholism, infection involving a toxin, nutritional deficiencies, physical injury, polyarteritis nodosa, systemic lupus erythematosus, Sjögren's syndrome, rheumatoid arthritis, sarcoidosis, Raynaud's disease, amyloidosis, Refsum's disease, Abetalipoproteinemia, Tangier disease, Krabe's disease, Metachromatic leukodystrophy, Fabry's disease. Dejerine-Sottas syndrome, and Charcot-Marie-Tooth Disease.

23. The method of claim 14, wherein said composition is administered to said patient topically on the skin.

24. The method of claim 14, wherein said spray is one or more of the group consisting of a pump spray, an aerosol spray, and a metered dose topical aerosol.

25. The method of claim 20, wherein said composition is administrable to said patient at about ½ to about 1/20 of a concentration of said nitric oxide donor required to induce vasodilation in healthy vasculature.
